(19) United States
(12) Patent Application Publication
(10) Pub. No.: US 2020/0061242 A1
(43) Pub. Date: Feb. 27, 2020

(54) TA2O5 AND TA2O5 - TIO2 HYBRID SURFACES FOR INVASIVE SURGICAL DEVICES

(71) Applicants: Jeffrey F. Roeder, Brookfield, CT (US); Peter C. Van Buskirk, Brookfield, CT (US)

(72) Inventors: Jeffrey F. Roeder, Brookfield, CT (US); Peter C. Van Buskirk, Brookfield, CT (US)

(21) Appl. No.: 16/552,890

(22) Filed: Aug. 27, 2019

Related U.S. Application Data
(63) Continuation-in-part of application No. 16/278,092, filed on Feb. 16, 2019.
(60) Provisional application No. 62/723,434, filed on Aug. 27, 2018.

(51) Int. Cl. A61L 27/30; A61L 27/06; A61L 27/04; A61L 27/18

(52) U.S. Cl. CPC A61L 27/306; A61L 27/06; A61L 27/047; A61L 2420/06; A61L 2300/404; A61L 2430/24; A61L 27/18

(57) ABSTRACT

A novel medical device thin film coating is disclosed. The coating may be produced by sputtering, CVD, MOCVD, or ALD. The conformal coating has dual antimicrobial and osseointegrative properties and comprises TiO₂ and Ta₂O₅ or intermediate phases. The TiO₂ provides antimicrobial properties via photocatalytic behavior in the visible or near visible light region and the Ta₂O₅ provides improved osseointegration. The TiO₂ and Ta₂O₅ may be doped with other cations. The coating combination may also be patterned into opened regions of one coating material on a continuous layer of the other coating material by using a partially decomposable organic patterning material between the layers.

### CROSS REFERENCE TO RELATED APPLICATIONS

This application is a U.S. Utility application taking priority from U.S. Provisional application No. 62/723,434, “Ta₂O₅ and Ta₂O₅—TiO₂ hybrid surfaces for invasive surgical devices,” filed Aug. 27, 2018. This application also takes priority from and incorporates fully by reference U.S. Utility application Ser. No. 16/278,092, filed Feb. 16, 2019.

### FIELD OF THE INVENTION

The present invention relates to joint implants and other invasive orthopedic devices with a surface modified to enhance osseointegration and to provide antimicrobial properties.

### BACKGROUND

Millions of joint replacements are performed every year. Periprosthetic joint infection (PJI) is a device-associated infection that poses a significant human and financial burden. Only a minority of joint arthroplasties become infected; however, these infections can cause significant morbidity, increase the risk of mortality, and contribute to a substantial proportion of health care expenditures. Treatment for PJIs usually involves multi-stage surgeries, which can lead to long hospital stays, delays in mobilization, pain, and large related costs per infection.

Systemic antibiotics are usually used to prevent or cure this type of infections. However; the effectiveness of antibiotics limited because they may not penetrate to the infection site and some pathogens have strong resistance to antibiotics.

The use of local antibiotics can result in decreased osseointegration. In addition, many strains of bacteria are becoming antibiotic resistant and difficult to treat. Further use of antibiotics exacerbates this problem.

The majority of PJIs are the result of microorganisms introduced to the exterior surface of the implant at the time of surgery through direct contact or aerosolized contamination. Planktonic microorganisms colonize the surface of the implant, and biofilm development begins. Biofilms are organized structures with numerous microorganisms surrounded by a self-produced matrix. Early biofilms are relatively unstable and still susceptible to host defense and antimicrobial agents. During biofilm maturation, a high density of microorganisms will form and provide the physiologic condition for microbial communication systems, i.e., quorum-sensing. Quorum-sensing regulates the production and release of various virulence factors protecting the biofilm from destruction. When organized in biofilms, microbes can be up to 1000-fold more resistant to antimicrobial drugs compared to planktonic microbes.

PJI is a representative example of the challenges posed by infections on invasive surgical devices, which generally refer to medical devices that come into contact in the body where bodily fluids are present. Of particular interest to the subject invention are invasive devices that are used in orthopedic applications. In these applications certain portions of the invasive device must have good adherence to bone. These may include artificial hip, knee, ankle and elbow implants. Further devices that would benefit from the subject invention include orthopedic plates, screws, pins, rods, and cages that may be used on any bone and dental implants. All of these devices are subject to contamination by planktonic bacteria, the formation of biofilms, and infection, and all include a least a portion of the device where strong adhesion to bone is required for satisfactory performance.

The formation of biofilms at the surface of medical devices is governed by the interactions among the device, the host, and the bacteria. Modification of the device surface provides a significant opportunity for preventing biofilm formation. To enhance the clinical outcome, the orthopedic implant material should prevent biofilm formation while at the same time possessing other beneficial properties, e.g, osteogenic properties. One such osteogenic property is osseointegration, or the integration of the bone with the implant to provide the desired biomechanical behavior for successful performance of the implant.

The surface characteristics of the implant highly affect the osseointegration. These characteristics include surface chemistry, topography, wettability, charge, surface energy, crystal structure and crystallinity, and roughness. Surface chemistry refers to the oxide present on Ti or other metal based alloys. In the case of Ti based alloys, the surface is a native layer of TiO₂ in the range of 3-10 nm. TiO₂ has good osseointegrative properties and low cytotoxicity. Surface topography has several scales. Macroscopic topography in the 0.1-3 mm range imparts a capability for strong bonding to bone in certain regions of an implanted device. Topography in the microscale (1-10 micron) and nanoscale range (1-500 nm) also impart antimicrobial and osseointegrative properties. Hydrophilic surfaces promote bone growth and anti-adhesion characteristics with respect to bacteria and biofilm formation. Charge characteristics affect initial bone-tissue interaction in the very early stages of osseointegration (e.g., on the order of seconds). Therefore, it is important that any coating have similar or improved charge characteristics. Surface energy is related to wettability. Hydrophilic surfaces have higher surface energies and a preference for water molecules compared to other molecules. As described herein, there are varying degrees of crystallinity and crystal phases that may be present on an invasive device surface. For the case of Ti based alloys, the native oxide is often amorphous. For optimal surface energy and wettability, crystalline phases are may be used to tune the surface energy and wettability. The anatase phase, alone or in combination with other crystalline phases including rutile and brookite are useful in this regard. Roughness corresponds to the surface topographies, and may be expressed as the highest roughness observed (Zmax) or the root mean squared average roughness (RMS roughness). Other feature characteristics may be described by their dimensions, e.g. height, width, diameter, etc., aspect ratio (ratio of height to diameter or width/thickness) and their spacing or pitch. Orthopedic surgery implants are commonly made of titanium (Ti) and its alloy with 6 wt % aluminum (Al) and 4 wt % vanadium (V), i.e., the Ti-6Al-4V alloy, both of which are bioinert and corrosion resistant, have a low Young's modulus, and most importantly are osteogenic. To achieve antimicrobial properties, researchers have investigated various strategies that focus on generating non-adhesive and/or bactericidal surfaces that can potentially prevent colonization or interrupt biofilm maturation, such as adding metal ions to implant materials and permanently binding antibiotics to implant surfaces. Drawbacks to these approaches include regulatory burdens, molecule instabilities, and local bacterial resistance.

Some current work is focused on surface nanostructures of the base implant material that interfere with bacterial adhesion and proliferation. These nanostructures include nanorods, nanoneedles, and nanocones. Some of the nanostructures mimic biological structures, e.g., cicada wing, gecko skin, etc. The nanostructures are on the order of 10s to 100s of nm in scale. Some of these features have high aspect ratios (ratio of height to width), e.g., 2:1 up to 5:1, which are susceptible to breakage when orthopedic devices are press fit into the bone. The process of placing the implant into the patient's bone often involves shear forces that break off high aspect ratio surface structures, especially in the case of hip implants. Breakage is a very large concern because small fragments broken off from the implant surface are uncontrolled foreign bodies within the patient that may migrate to unanticipated areas. For example, they could migrate to the moving areas of the joint where they could interfere with the mechanical operation or damage wear surfaces or cause inflammation of tissue.

One lithographic method that has been used to create the patterned structures of TiO₂ on a surface is the use of a block copolymer (BCP). BCPs produce discrete regions of different polymer phases that may have a variety of geometric patterns based on the BCP and the ratio of its constituent polymers. The pattern formation typically occurs over a period of time as the block polymer self-segregates. The geometric patterns may be regions of one polymer phase dispersed in the other as islands, or alternating bands of phases, i.e., lamellae. An example of a suitable BCP is polystyrene-polymethylmethacrylate (PS-b-PMMA). BCPs have been shown to allow deposition of materials like TiO₂ in selected areas. For example, using TiCl₄ as an ALD precursor results in selective deposition of TiO₂ on PMMA regions with little or no deposition on the PS regions when the BCP is disposed on the surface of a substrate. After selective film deposition, the BCP is removed, typically by an oxygen ashing process. A TiO₂/polymer aggregate remains in the former locations of the PMMA and no TiO₂ remains where the PS was located. The TiO₂/polymer aggregate may be further consolidated and purified by heat treatments, but it may be difficult to fully remove the polymer, which may lead to carbon contamination.

The surface of Ti-containing implant devices is TiO₂, a natively formed oxide that can be photocatalytic. One advantage of TiO₂ is that the layer may be photoactive, forming reactive oxygen species (ROS) from oxygen or moisture under illumination. ROS created during photocatalysis are broadly antimicrobial, behaving like hydrogen peroxide. At present, the TiO₂ layers are typically not activated by visible light, but by UV light, typically UVC (255-280 nm wavelength), which is harmful the operating room personnel. It would accordingly be a great advantage to have a TiO₂ layer that may be stimulated by light in the visible spectrum to create ROS, or near visible spectrum, e.g, violet or UVA (320-400 nm) as described in U.S. Patent Application 62/632,312, which is incorporated in its entirely by reference.

Antimicrobial properties may arise from more than one property of a surface. Surfaces to which bacterial are less likely to adhere are antimicrobial because the bacteria cannot form colonies that can further organize into biofilms. Surfaces can also be bactericidal meaning that they have properties that kill bacteria. The photocatalytic surfaces described in this patent are bactericidal because the ROS attack the cell calls of the bacteria and destroy their ability to survive or replicate. They may also inhibit adherence of bacteria. Both anti-adhesion and bactericidal actions are antimicrobial. Surface topographies may be antimicrobial or bactericidal, depending on the species of bacteria, the strain of the bacteria, and the feature sizes.

Accordingly, it would be an advantageous improvement to have a photocatalytic surface on invasive surgical devices that produces an antimicrobial effect, which may be bactericidal, is non-toxic, and is compatible with other intentional surface nanostructures along with appropriate systems for illumination, and other ancillary materials and systems, including those for maintaining sterility, activating the antimicrobial surface, potentiating its antimicrobial properties, facilitating its remaining active while exposed to the operating room environment.

Invasive orthopedic devices typically comprise polymers or several metal alloys for different parts of the device. For example, a hip implant has a stem and a ball on one side and a cup on the other side. Stems are typically fabricated from titanium (Ti) or Ti-6Al-4V. The ball and bearing surface of the cup are often Co—Cr alloys that have superior resistance to wear compared to the Ti alloy. The back side of the cup may also comprise Ti or a Ti alloy, along with Ti based fixing screws. A polymeric material is typically disposed between the ball and the cup to provide lubricity. Other devices may be formed entirely from a polymer. For example, spine cages, may be formed from polyetheretherketone (PEEK).

The surface of the Ti based stem may roughened in certain areas to promote osseointegration, or the development of strong bone tissue attachment to the device. This may be performed by bead or grit blasting, or by additive processes such as thermal or plasma spray. The surface of the stem in both roughened and non-roughened areas has a TiO₂ layer formed by various wet treatments (e.g., acid cleans), aqueous rinses, and or thermal treatments. Surfaces of other implant materials (e.g., metal alloys other than Ti-based, i.e., stainless steel, as well as polymers) may be roughened by various means, including abrasion, grit blasting, and plasma etching.

It is known that a Ta surface layer applied to the surface of an invasive Ti device further promotes osseointegration. Such layers may be formed by physical vapor deposition methods, e.g., evaporation, sputtering, or thermal or plasma spray. In practice, the outermost layer is Ta₂O₅ which forms due to highly reactive nature of Ta with ambient oxygen in a manner similar to the formation of a native TiO₂ layer on Ti. It would be a further advantage to have the combination of the superior osseointegrating properties of Ta₂O₅ combined with the antimicrobial properties of TiO₂ on smooth, roughend, or highly porous/torturous surfaces of invasive orthopedic devices as desired by a particular medical indication or anatomical use. Such surfaces may be metal, ceramic, or polymeric as determined by the device designer. It would be yet another advantage to provide surface structures comprising Ta₂O₅ and TiO₂ that have aspect ratios of 1:1 or lower to improve the ability of the structure to survive the implantation process into the bone.

### SUMMARY OF THE INVENTION

The present invention relates to a conformal coating of Ta₂O₅ or a TiO₂—Ta₂O₅ alloy disposed on the surface of an invasive orthopedic device that provides superior osseointgration and antimicrobial properties. The TiO₂—Ta₂O₅ alloy may be a homogeneous single phase, or a multiphase mixture, or may be comprised of one constituent disposed on top of the other, with the top constituent in a discontinuous and/or intentionally patterned layer.

### BRIEF DESCRIPTION OF THE DRAWINGS

FIG. 1 is a schematic of a hip implant.

FIG. 2 is a schematic of a conformal coating comprising a homogeneous film on the surface of the hip implant.

FIG. 3 is a schematic of a conformal coating comprising a mixture TiO₂ and Ta₂O₅ on the surface of the hip implant with discrete regions of Ti-rich and Ta-rich compositions.

FIG. 4 is a process flow for forming discrete regions of one constituent on top of another where the lower layer is TiO₂.

FIG. 5 is a schematic of a device with a surface layer comprising TiO₂ with discrete regions of Ta₂O₅ disposed on top of it.

FIG. 6 is a schematic of a device with a surface layer comprising Ta₂O₅ with discrete regions of TiO₂ disposed on top of it.

FIG. 7 is a schematic of a spherical bacterium on a device with a surface layer comprising TiO₂ and discrete regions of Ta₂O₅ disposed on top of it.

FIG. 8 is a plot of the required spacing of a pair of features on a surface that allow contact with a sphere to the surface for given height of the features.

### DETAILED DESCRIPTION OF THE INVENTION, AND PREFERRED EMBODIMENTS THEREOF

The present invention relates to a conformal coating of TiO₂ and Ta₂O₅ disposed on the surface of an invasive orthopedic device that provides superior osseointgration and antimicrobial properties. While the coatings herein are described as on a surface, they need not be in direct contact with the surface, intermediate layers could lie between the surface of the invasive medical device and the coatings of the subject invention.

In one aspect the invention relates to the formation of a homogeneous alloy of TiO₂ and Ta₂O₅ formed on the surface of an invasive orthopedic device. The homogenous layer provides improved osseintegration properties combined with photocatalytic behavior from the TiO₂ which provides antimicrobial or bacteriostatic properties.

In another aspect, the invention relates to the formation of regions of TiO₂ and Ta₂O₅ in a layer formed on the surface of an invasive orthopedic device. The Ta₂O₅ layer provides improved osseintegration properties combined with photocatalytic behavior from the TiO₂ which provides antimicrobial or bacteriostatic properties.

In yet another aspect, the invention relates to the formation of a layer comprising TiO₂ or Ta₂O₅ or an alloy thereof, with a second discontinuous layer of one of the two constituents disposed on top of the first layer. The discontinuous layer has a discontinuous morphology, which may alternatively be described as discrete regions TiO₂ or Ta₂O₅, with spaces or intervals between the discrete regions. The Ta₂O₅ provides improved osseintegration properties combined with photocatalytic behavior from the TiO₂ which provides antimicrobial or bacteriostatic properties. Further, the feature sizes of the regions within the discontinuous layer may provide antimicrobial properties by virtue of their geometric shape and spacings, which may reduce adhesion of bacteria that can form biofilms.

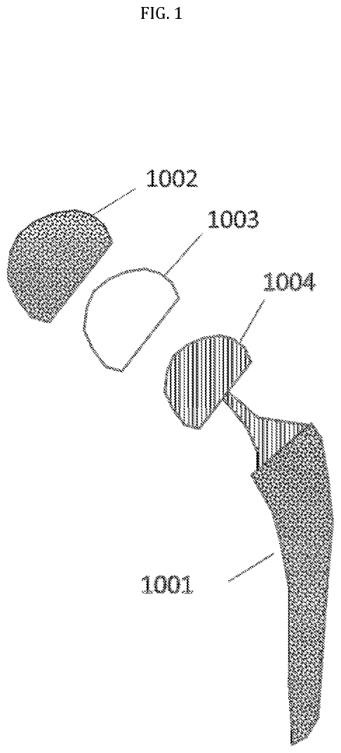

An example orthopedic device, a human hip implant, is shown in FIG. 1. A typical device has a Ti alloy stem 1001 that is implanted into the femur and a cup 1002 that is implanted into the pelvis. A ball 1003 attaches to the stem 1001 and fits into the cup 1002 to provide articulation of the joint. A polymeric liner 1004 may be placed between the ball and the cup.

In one aspect, the invention relates to coating the stem 1001 and back (bone) side of the acetabular cup 1002. These surfaces are often intentionally roughened to promote integration into bone. The surface roughness may be on several length scales, including millimeter, micrometer (micron), and nanometer. Each length scale may play a particular biomechanical or biological role. For example, millimeter scale roughness provides enhanced bone/device strength as the bone grows around the asperities. Micrometer scale roughness may provide similar functionality. Nanometer scale features may provide antimicrobial properties that assist in preventing periprosthetic joint infections (PJIs). One objective of the present invention is to provide a thin film coating that promotes osseointegration and also provides additional antimicrobial properties to prevent PJIs. In order not to disturb the antimicrobial properties of the intentionally structured features, the coating should provide high fidelity the surface, i.e., the coating should be highly conformal. Conformality may be described as the ratio of the thinnest region of a coating to the thickest region as it traverses a non-planar surface. A perfectly conformal film would have a conformality of 1, which may also be expressed as a percentage of 100%. A slightly less conformal film may have a conformality of 0.95, or 95%. A less conformal film may have a conformality of 0.90, or 90% and so on.

Several vacuum coating methods provide various degrees of conformality. For example, sputtering can provide a degree of conformality. For a simple undulating surface, conformality of sputtering may be in the region of 90%. For a three dimensional surface where the back side of the feature is completely out of the line of sight of the sputtering source, the conformality may approach 0. Sputtering can be carried out via radio frequency (RF) magnetron or direct current (DC) magnetron techniques. In order the create a mixed alloy, Ti and Ta may be co-sputtered from dual targets or from composite targets that may be an alloy or a target with different regions of Ti and Ta. The target may comprise pure metals where the oxide coating is formed by reaction with a controlled amount of oxygen in the vacuum ambient.

Chemical vapor deposition (CVD) or metalorganic chemical vapor deposition (MOCVD) may also be used to create conformal coatings. In general, the conformality of CVD and MOCVD are better than sputtering and may approach 100% in some cases. CVD and MOCVD may be carried out in temperature regions where the process is kinetically limited by surface reactions (lower temperatures) or by mass transport near the surface (higher temperatures). CVD and MOCVD at higher temperatures may produce a more crystalline film compared to CVD or MOCVD at lower temperatures. In CVD and MOCVD, the metal precursor is normally simultaneously introduced with the co-reactant into a deposition chamber where the substrate is held at elevated temperature. For oxides, the co-reactant may be oxygen, nitrous oxide, oxygen plasma or other oxygen containing moieties. The deposition chamber may be held a pressure below atmospheric pressure. The chamber pressure may be below 10 Torr. Alternatively, the chamber pressure may be below 2 Torr. The oxidizer may also be modulated in an alternating sequence with the metal precursor.

Atomic layer deposition (ALD) has the capability to produce highly conformal films over very challenging features with large aspect ratios (e.g. depth to width of a trench or hole) or on complex three dimensional (3D) shapes. Conformality may approach 100% in many cases. ALD employs alternating exposures of a precursor for the metal, and in the case of oxide film formation, an oxidizer. Suitable oxidizers include water, ozone, and oxygen plasma. The exposures of precursor and oxidizer are separated by inert gas purges that keep the reactants temporally separated and limit the reaction to the surface. In principle, monolayer surface coverage is achievable with the metal precursor. The film is built up in repeating cycles of precursor dose—inert purge—oxidizer dose—inert purge. Each such sequence is known as an “ALD cycle”. Thin films produced by ALD may have extremely high (>99%) conformality and freedom from defects such as pin holes.

Precursors useful for CVD, MOCVD, and ALD of titanium oxide include a number of inorganic and metalorganic compounds, respectively. Inorganic compounds useful for CVD and ALD of titanium oxide include TiCl₄, TiBr₄ and TiI₄. Metalorganic precursors for ALD and MOCVD titanium oxide include ketonates, iminates, alkoxides, amides, cyclopentatdienyls, amidinates, and guanidinates, some of which are fluorine containing. Mixed ligand precursors also exist. Examples of Ti precursors include Ti(OiPr)₂(thd)₂, Ti(NMe₂)₄,.Ti(NEt₂)₄, Ti(NEtMe)₄ where OiPr and thd represent isopropoxide and tetramethaneheptanedianoto ligands, respectively and Et and Me represent ethyl and methyl. Metalorganic precursors for MOCVD and ALD of tantualum oxide include Ta(OEt)₅, Ta(NMe₂)₅, Ta(NEt₂)₅, Ta(NEtMe)₅ and other variants including ring type compounds with N or C bridging between ligands. Amide-imide mixed ligand precursors, e.g. tantalum triamide-imide may be used. Inorganic halides of Ta, e.g., TaCl₅, TaF₅, TaBr₅ and TaI₅ may be used for CVD or ALD of tantalum oxide. ALD of TiO₂ using Ti(NMe₂)₄ and water at a temperature of 250° C. yields a growth rate of ˜0.5 Å/cycle. ALD of Ta₂O₅ using Ta(NMe₂)₅ and water at a temperature of 250° C. yields a growth rate of ˜0.8 Å/cycle.

MOCVD or ALD may be carried out with solid sources held in bubblers through which a carrier gas is flowed to convey the source to the deposition chamber. The sources may also be dissolved in an organic solvent as individual sources or combined together. Key criteria of a solvent system are (1) high boiling point to reduce the chance of flash off of the solvent, (2) high solubility for the compound, (3) low cost. Useful hydrocarbon solvents may include, for example: octane, decane, isopropanol, cyclohexane, tetrahydrofuran, and butyl acetate or mixtures comprising these and other organic solvents. Lewis base adducts may also be incorporated as additions to the solvent(s) for beneficial effects on solubility and to prevent possible oligimerization of the precursor molecules. Examples of useful Lewis Bases include polyamines polyethers, crown ethers, and the like. Pentamethylenediamine is a one example of a polyamine. Examples of polyethers include various glymes such as mono-, di-, tri-, and tetraglyme.

In another aspect, it is desirable to control the composition and phases of the deposited film. The film may range from nearly pure (99.9%) TiO₂ to pure (100%) Ta₂O₅ with respect to TiO₂. The film may be deposited in discrete layers of TiO₂ or Ta₂O₅ using CVD, MOCVD or ALD. The film may also be deposited as a mixed alloy of the two metal cations (Ti and Ta) and oxygen. In order to carry out the mixed alloy route, precursors for Ti and Ta should have similar or identical ligands to prevent undesired exchange mechanisms in the gas phase that could result in formation of particles or low volatility materials.

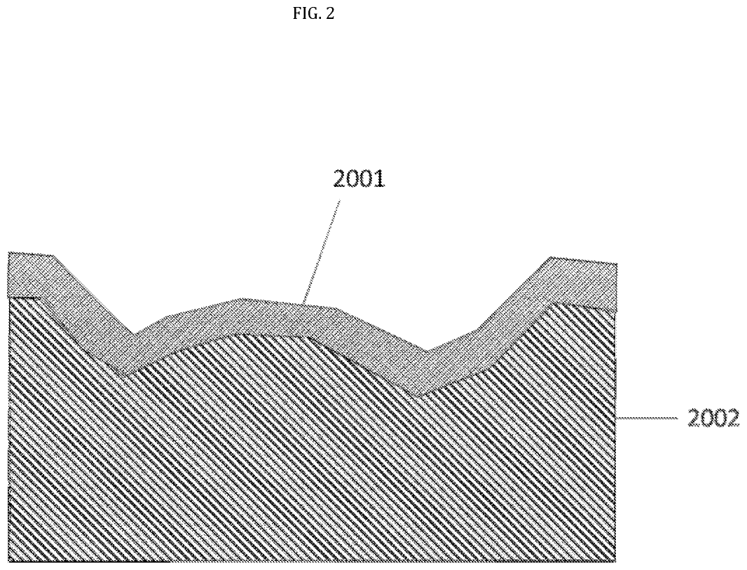

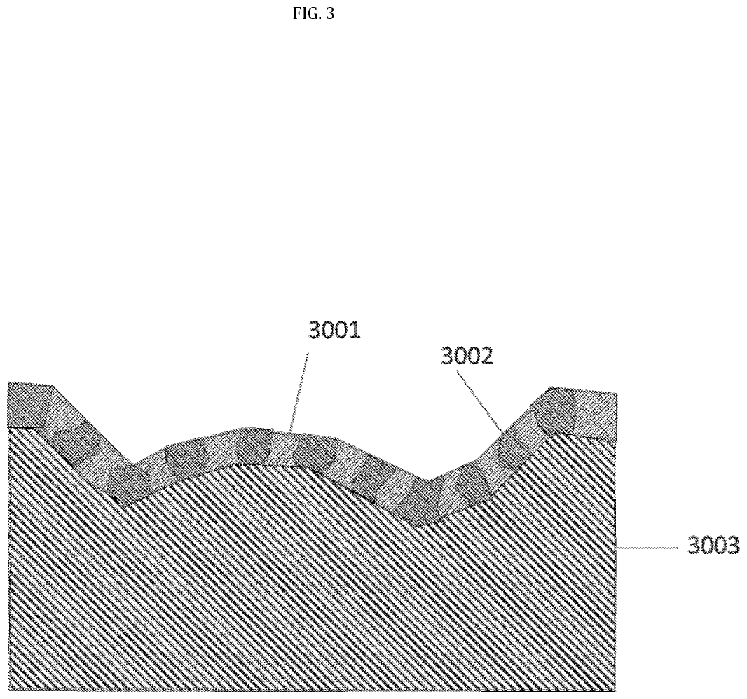

In one embodiment, a homogeneous TiO₂—Ta₂O₅ film 2001 may be deposited onto the surface of a medical device 2002 using sputtering, MOCVD or ALD as a film of uniform composition (FIG. 2). Films thickness may vary from 10-1000 nm, more preferably from 40-150 nm. The film may then be heat treated to cause phase separation (FIG. 3) resulting in composition phase regions that are Ti-rich 3001 and Ta-rich 3002. As defined herein, Ti-rich refers to compositions of TiO₂-13 Ta₂O₅ where the average content of TiO₂ among all phases present is >50 atomic % (at %) TiO₂ and Ta-rich to compositions of TiO₂-Ta₂O₅ where the average content of Ta₂O₅ among all phases present is >50 atomic % (at %) Ta₂O₅. This is to say that each phase region may be a homogenous single phase or a region of multiple phases with a distinct average composition different from a neighboring region. Depending on the temperature of the heat treatment, the phase regions may be smaller or larger in size. Smaller phase regions result for shorter or lower temperature heat treatments. Heat treatment may occur in an oxygen containing ambient, e.g., air in a temperature range from 200-600° C., more preferably in a temperature range of 400-600° C. Heat treatment times may range from 10 minutes to 3 hours. These films may have conformalities of >80%, more preferably 90%, and most preferably 95%.

The TiO₂—Ta₂O₅ film may be deposited onto the entire surface of the device or a portion thereof. For example, on a hip stem, the film may be deposited everywhere but the taper that is fit into the ball, or it may be deposited on the entire device. Preferably, the film is deposited onto all surfaces of the stem with the exception of the taper. For a screw, it may be deposited everywhere except the head, or it may be deposited on the entire screw.

The phases formed by heat treatment may comprise anatase, rutile, or brookite phases of TiO₂, and orthorhombic β-Ta₂O₅ or hexagonal δ-Ta₂O₅. The Ta₂O₅ may also be amorphous prior to or after heat treatment. An intermediate phase of TiTa₂O₇ may also be formed, depending on film composition and heat treatment, alone or in combination with Ti-rich or Ta-rich oxides. The areal percentage of the TiO₂-rich to Ta₂O₅-rich segregated phases at the surface for segregated phases may be from 0-99.9%

In another process the TiO₂ and Ta₂O₅ regions may be formed in alternating layers to form a nanolaminate. This nanolaminate may be heat treated or annealed to segregate Ti-rich and Ta-rich regions as depicted in FIG. 3. Heat treatment may occur in an oxygen containing ambient, e.g., air in a temperature range from 200-600° C., more preferably in a temperature range of 400-600° C. Heat treatment times may range from 10 minutes to 3 hours. These films may have conformalities of >80%, more preferably 90%, and most preferably 95%.

In another aspect, the thin film coatings may be further modified by the introduction of other dopants, either cation or anion in nature in order to shift the bandgap of the Ti-rich phase(s) to produce photocatalytic behavior in the visible or near visible light region. Cation dopants may be cerium and anion dopants may be nitrogen. Metalorganic precursors for cerium include a number of metalorganic compounds, including ketonates, iminates, alkoxides, amides, amidinates, and guanidinates, some of which are fluorine containing. In general, amidinates and guanidinates are useful for ALD, as are cyclopentadienyls. Mixed ligand cyclopentadienyl-amidinate precursors also exist for Ce, e.g. Ce(iPrCp)₂-(isopropylamindinate). Nitrogen may be introduced through nitrogen containing ligands in the metalorganic precursor or via co-reactants, e.g., ammonia or nitrogen in the sputtering environment, ammonia or other nitrogen containing precursors in the MOCVD or ALD environment. For ALD, nitrogen containing reagents like pyridine or urea may be used in combination with water.

In addition to having an antimicrobial effect via photocatalysis, the applied coatings may be hydrophilic, which may further enhance osseointegration. The hydrophilic surface may also have the property of reducing the adherence of bacteria, thereby reducing the propensity for the formation of biofilms from colonies forming units of bacteria. The hydrophilicity of the surface is measured by wetting angle with a water droplet. Hydrophilic surfaces have a contact angle of less than 90 degrees.

The antimicrobial properties may comprise antibacterial, bactericidal, antiviral, fungicidal and sporicidal functions.

In addition to stems, other components of invasive devices may be coated as described below. These components may comprise balls, cups, polymeric components, screws, plates, fixing devices, screens, mesh, porous metals, and the like.

In another embodiment, the coating may provide the topological features that provide antimicrobial properties by virtue of their specific geometries. This may be accomplished by a combination of lithographic methods and selective deposition to form novel structures with enhanced osseointegrative properties combined with antimicrobial properties. These methods may be applied to flat or curved surfaces, or even porous surfaces.

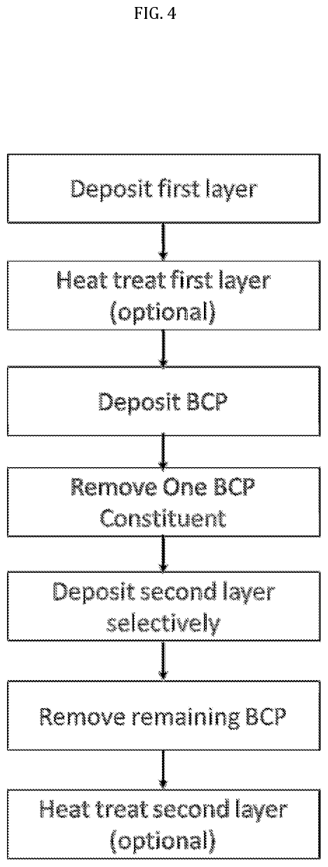

In another embodiment, a block copolymer (BCP), e.g., PS-b-PMMA, is used in a different manner from the prior art. In order to produce a layer of one constituent (i.e., TiO₂ or Ta₂O₅) with a second discontinuous layer of the other constituent disposed on it, a fabrication sequence described in FIG. 4 may be used. The first layer is formed or deposited followed by an optional heat treatment to produce the desired phase (or phases) if not already formed by the deposition or formation process. Deposition processes include sputtering, ion beam deposition, CVD, MOCVD, and ALD, which may be thermal or plasma assisted. Formation processes include annealing and anodizing. In the case of a TiO₂ layer, the TiO₂ may include anatase TiO₂, rutile TiO₂, or brookite TiO₂, or combinations thereof. One preferred combination is anatase and rutile, with a fraction of anatase greater than 50%. A BCP, e.g., PS-b-PMMA is then applied either as a neat liquid or dissolved in a suitable solvent. Curing of the BCP may be carried out at elevated temperature above the glass transition temperature of the constituents, either in the presence of, or without solvent vapor, at atmospheric or vacuum conditions. Temperatures in the range of 150-200° C. may be used, more preferably 160-180° C. After the BCP has cured, one phase is removed, e.g., PMMA, leaving regions of PS on the surface of the first layer that form a mask. The shape of the regions is controlled by the ratio of one monomer to the other in the BCP. The PMMA may be removed by UV illumination or by chemical means. Chemical means include acetic acid, which preferentially dissolves PMMA compared to PS. The second constituent is now formed by selective deposition on the patterned surface using a vapor chemical deposition method, e.g. CVD or ALD. Selective deposition occurs on the first layer but not to a significant extent on the PS. After deposition of the second constituent, the remaining component of the BCP may be removed, e.g., via oxygen ashing or a solvent. The term “significant extent” relating to deposition on PS means that the PS may be effectively removed to expose the underlying surface, i.e., the top surface of the first layer between the regions of the patterned second layer. The selectively deposited film may optionally be heat treated to form a desired phase.

It is also disclosed that the BCP may be used for subtractive etching of one layer disposed on top of another layer, e.g., TiO₂ may be the first layer and Ta₂O₅ may be the second layer or vice-versa, and a selective etch is used to remove the upper layer beneath the pattern provided by a BCP from which one phase has been preferentially or selectively removed. This method may also be used to create features of a single layer on a substrate, for example TiO₂ deposited on Ta metal where the Ta metal has a Ta₂O₅ surface layer. Another example could be the formation of a patterned layer of TiO₂ or Ta₂O₅ on PEEK or another structural orthopedic polymer. A continuous layer of TiO₂ or Ta₂O₅ may be deposited on the structural orthopedic polymer prior to application of the BCP.

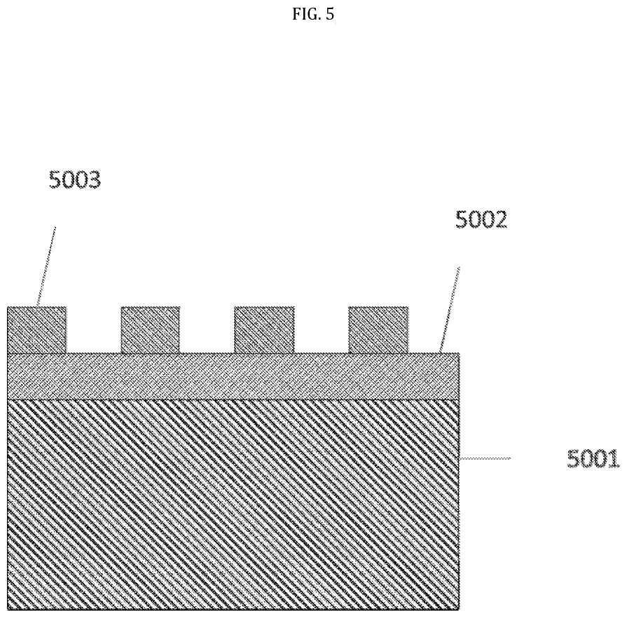

In one embodiment, a TiO₂ layer is formed on a substrate. The substrate may be a metal, a ceramic, or a polymer. Examples of metals include Ti, Ti-6Al-4V, tantalum, and other medical Ti and Ta alloys. Examples of ceramics include zirconia, yttria stabilized zirconia, and alumina. Examples of polymers include PEEK. In the case of a Ti containing substrate, the TiO₂ layer may be formed by anodization or more generally on any substrate by a vapor phase method such as CVD, MOCVD, ALD, or sputtering. A preferred method is ALD using a precursor selected from those described previously. The TiO₂ phase may be anatase, brookite, or rutile, or combinations of the same. The preferred phase mixture is rutile and anatase. The ratio of rutile to anatase may be from 10:1 to 1:10 by volume. The preferred ratio is between 3:7 and 7:3. The thickness of the TiO₂ layer may be from 1-100 nm, preferably 20-80 nm and most preferably 25-60 nm. The TiO₂ layer may be annealed between 300-600° C. to adjust the phase ratio, e.g., rutile to anatase. A BCP is applied to the TiO₂ layer and cured. The BCP is PS-b-PMMA. The PMMA portion of the BCP is removed using a solvent that attacks C═O bonds in the PMMA. Ta₂O₅ is then selectively deposited on the exposed TiO₂ layer by ALD using precursor(s) described earlier. The thickness of the Ta₂O₅layer may be between 0.5-20 nm, preferably 0.5-10 nm, more preferably 0.5-2 nm. After deposition, the remaining BCP (i.e., PS) is removed by ashing. The resulting structure is shown in cross-section in FIG. 5: a substrate 5001 with a TiO₂ layer 5002 and Ta₂O₅ regions 5003 on the surface. The Ta₂O₅ regions form a discontinuous layer, i.e., they are separated by spaces from one another in one or two dimensions within a plane parallel to the plane of the first layer. The width of the Ta₂O₅ regions may be from 10-1000 nm and the spacing may be from 10-1000 nm. The width and spacing are preferably 20-100 nm. Note that these widths and spaces are averages, not every single space or width must fall within the range. In addition, by using the phrase “separated by spaces” the regions of the discontinuous layer may be completely separated by spaces or island-like, or alternatively the regions of the discontinuous layer may be connected with spaces between the connected regions, like a honeycomb, or may be a mixture of the two types of connectivity. While the regions of the discontinuous layer may be completely separated by spaces, this invention is not intended to cover separately formed particles of material which are attached to the continuous layer, but rather an actual deposited, patterned layer. When viewed from above, the Ta₂O₅ regions may be equiaxed (e.g., circular, hexagonal, or the like) or lamellar. The TiO₂ may be doped with Ta, Ce, or N.

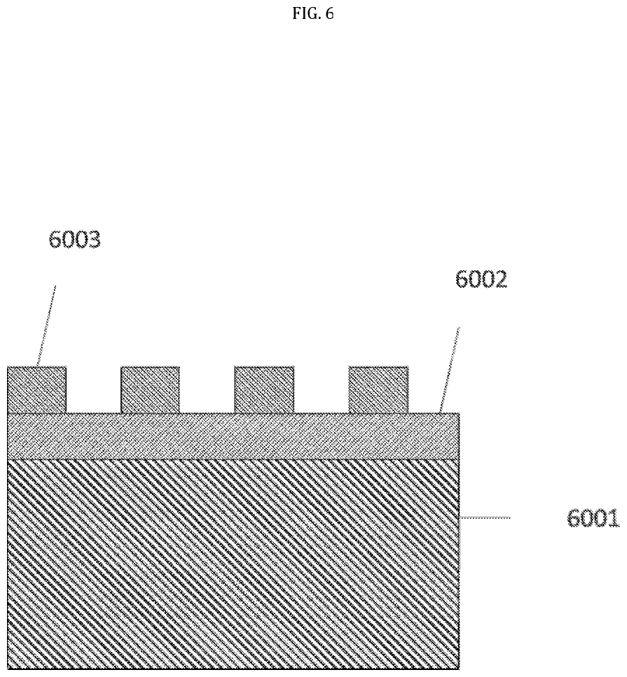

In another embodiment, a Ta₂O₅layer is formed on a substrate. The substrate may be a metal, a ceramic, or a polymer. Examples of metals include Ti, Ti-6Al-4V, tantalum, and other medical Ti and Ta alloys. Examples of ceramics include zirconia, yttria stabilized zirconia, and alumina. Examples of polymers include PEEK. In the case of a Ta containing substrate, the Ta₂O₅layer may be formed by anodization or more generally on any substrate by a vapor phase method such as MOCVD, ALD, or sputtering. The Ta₂O₅ layer may also be a native oxide. A preferred method is ALD using a precursor selected from those described previously. The Ta₂O₅ phase may be amorphous or crystalline or combinations thereof. The thickness of the Ta₂O₅layer may be from 1-100 nm, preferably 20-80 nm and most preferably 25-60 nm. The Ta₂O₅ layer may be annealed between 300-600° C. to adjust the phase ratio, e.g., amorphous to crystalline. The ratio of amorphous to crystalline material may range from 1:100 to 100:1. A BCP is applied to the Ta₂O₅ layer and cured. The BCP is PS-b-PMMA. The PMMA portion of the BCP is removed using a solvent that attacks C═O bonds in the PMMA. TiO₂ is then selectively deposited on the exposed Ta₂O₅ layer by ALD using precursor(s) described earlier. The thickness of the TiO₂layer may be between 1-100 nm, preferably 10-80 nm, more preferably 10-50 nm. After deposition, the remaining BCP (i.e., PS) is removed by ashing. The resulting structure is shown in cross-section in FIG. 6: a substrate 6001 with a Ta₂O₅layer 6002 and TiO₂ regions 6003 on the surface. The TiO₂ regions are discontinuous, i.e., they are separated by spaces from one another in one or two dimensions within a plane parallel to the plane of the first layer. The width of the TiO₂ regions may be from 10-1000 nm and the spacing may be from 10-1000 nm. The width and spacing are preferably 20-100 nm. When viewed from above, the TiO₂ regions may be equiaxed (e.g., circular, hexagonal, or the like) or lamellar. The TiO₂ may be doped with Ta, Ce, or N.

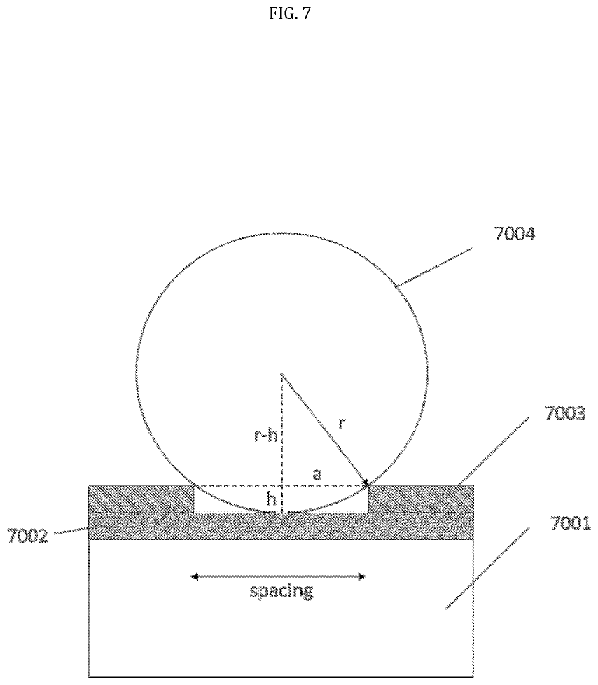

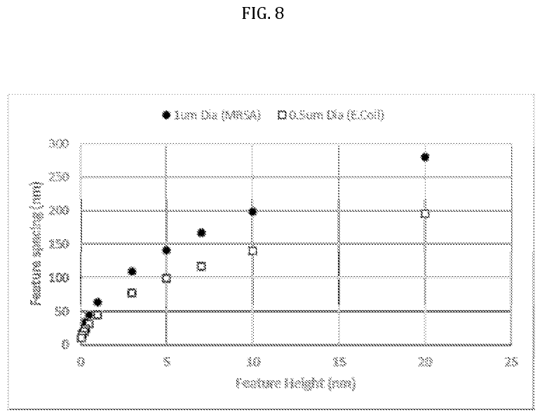

Regarding the preferred thickness of the embodiment of Ta₂O₅ regions on TiO₂, a simple model may be used to estimate the desired height and spacing of the Ta₂O₅ regions. Many of the bacteria of interest are spheroidal, ellipsoidal, or rod shaped. For example, methicillin resistant Staphylococcus aureus (MRSA) is spheroidal, with a diameter of approximately 1 micron. Eschericia coli (E. coli) is rod shaped with a diameter of approximately 0.5 micron and a length of approximately 2 microns. Enterococcus varies ellipsoidal with an average diameter of approximately 0.5 to 2 microns. Pseudomonas aeruginosa is rod like with a diameter of approximately 0.65 microns and a length of 2.25 microns. A substrate 7001 with a TiO₂ layer 7002 and Ta₂O₅ regions 7003 is shown in FIG. 7. A spherical representation of a bacterium 7004 is shown in contact with the surface of the TiO₂ layer 7002 between two Ta₂O₅ regions 7003. For optimal bactericidal action of the surface, the bacterium 7004 should be able to approach the surface of the TiO₂ layer 7002 with close proximity so that ROS created at the surface by light illumination can interact with the bacterial cell wall. A simple geometric model can be used to determine the spacing between features that permits contact of a bacterium represented as a sphere. Referring to FIG. 7, simple geometry defines the relationship between the spacing (=2a) and the radius (r) of the bacterium 7004 with the constraint that the height (h) of the Ta₂O₅ regions 7003 should be the maximum to allow contact of the sphere with the surface. The relationship between these parameters is r²=(r−h)² +a², which may be rearranged to a (half the spacing)=(2rh−h²)^{1/2}. A plot of minimum spacing for contact of a spherical section of a bacterium of diameters 1 micron and 0.5 micron with a surface between two features with height (h) is shown in FIG. 8. It can be seen that the ideal height is considerably smaller than the spacing. For feature sizes where the width of the feature is on the order of 40 nm, this gives a preferred height of the features to be approximately 1 nm. Given that bacterium are not rigid spheres and have deformability and that the bacterial wall need only come into contact with the ROS which will be in proximity to the surface of the TiO₂ layer 7002, this delineates a preferred range of the height of the features to be 0.5-2 nm. In this example, the features are the Ta₂O₅ regions 7003. We note that for a 40 nm wide Ta₂O₅ region with lnm height, the aspect ratio of the Ta₂O₅ region 7003 is 0.025:1 which is much less than an aspect ratio of 1:1, and which will have favorable mechanical resistance to shear forces from a geometric perspective.

### EXAMPLES

Example 1: An invasive surgical device is placed in a vacuum deposition chamber at a reduced pressure of 1 Torr and exposed to a mixture of Ti(NMe₂)₄ and Ta(NMe₂)₅ in a ratio chosen to result in a ratio of TiO₂:Ta₂O₅ of 50:1 in the film. The device is held at a temperature of 350° C. in contact with the precursors and oxygen for a time sufficient to form a film of TiO₂—Ta₂O₅ on the surface of the device of at least 50 nm. Preferably the film thickness is 50-75 nm. Optionally, the device is post-annealed in air at 600° C. for at least 10 minutes. The resulting film has an average composition of 98 at % TiO₂-2 at % Ta₂O₅+/−2 at % TiO₂.

Example 2: An invasive surgical device is placed in a vacuum deposition chamber at a reduced pressure of 1 Torr and exposed to a mixture of Ti(NMe₂)₄ and Ta(NMe₂)₅ in a ratio chosen to result in a ratio of TiO₂:Ta₂O₅ of 20:1 in the film. The device is held at a temperature of 350° C. in contact with the precursors and oxygen for a time sufficient to form a film of TiO₂—Ta₂O₅ on the surface of the device of at least 50 nm. Preferably the film thickness is 50-75 nm. Optionally, the device is post-annealed in air at 600° C. for at least 10 minutes. The resulting film has an average composition of 95 at % TiO₂-5 at % Ta₂O₅+/−2 at % TiO₂.

Example 3: An invasive surgical device is placed in a vacuum deposition chamber at a reduced pressure of 1 Torr and exposed to a mixture of Ti(NMe₂)₄ and Ta(NMe₂)₅ in a ratio chosen to result in a ratio of TiO₂:Ta₂O₅ of 10:1 in the film. The device is held at a temperature of 350° C. in contact with the precursors and oxygen for a time sufficient to form a film of TiO₂—Ta₂O₅ on the surface of the device of at least 50 nm. Preferably the film thickness is 50-75 nm. Optionally, the device is post-annealed in air at 600° C. for at least 10 minutes. The resulting film has an average composition of 91 at % TiO₂-9 at % Ta₂O₅+/−2 at % TiO₂.

Example 4: An invasive surgical device is placed in a vacuum deposition chamber at a reduced pressure of 1 Torr and exposed to a mixture of Ti(NMe₂)₄ and Ta(NMe₂)₅ in a ratio chosen to result in a ratio of TiO₂:Ta₂O₅ of 5:1 in the film. The device is held at a temperature of 350° C. in contact with the precursors and oxygen for a time sufficient to form a film of TiO₂—Ta₂O₅ on the surface of the device of at least 50 nm. Preferably the film thickness is 50-75 nm. Optionally, the device is post-annealed in air at 600° C. for at least 10 minutes. The resulting film has an average composition of 83 at % TiO₂-17 at % Ta₂O₅+/−2 at % TiO₂.

Example 5: An invasive surgical device is placed in a vacuum deposition chamber at a reduced pressure of 1 Torr and exposed to a mixture of Ti(NMe₂)₄ and Ta(NMe₂)₅ in a ratio chosen to result in a ratio of TiO₂:Ta₂O₅ of 1:1 in the film. The device is held at a temperature of 350° C. in contact with the precursors and oxygen for a time sufficient to form a film of TiO₂—Ta₂O₅ on the surface of the device of at least 50 nm. Preferably the film thickness is 50-75 nm. Optionally, the device is post-annealed in air at 600° C. for at least 10 minutes. The resulting film has an average composition of 50 at % TiO₂-50 at % Ta₂O₅+/−2 at % TiO₂.

Example 6: An invasive surgical device is placed in a vacuum deposition chamber at a reduced pressure of 1 Torr and exposed to a mixture of Ti(NMe₂)₄ and Ta(NMe₂)₅ in a ratio a ratio chosen to result in a ratio of TiO₂:Ta₂O₅ of 1:5 in the film. The device is held at a temperature of 350° C. in contact with the precursors and oxygen for a time sufficient to form a film of TiO₂—Ta₂O₅ on the surface of the device of at least 50 nm. Preferably the thickness is 50-75 nm. Optionally, the device is post-annealed in air at 600° C. for at least 10 minutes. The resulting film has an average composition of 17 at % TiO₂-83 at % Ta₂O₅+/−2 at % TiO₂.

Example 7: An invasive surgical device is placed in a vacuum deposition chamber at a reduced pressure of 1 Torr and exposed to a mixture of Ti(NMe₂)₄ and Ta(NMe₂)₅ in a ratio a ratio chosen to result in a ratio of TiO₂:Ta₂O₅ of 1:50 in the film. The device is held at a temperature of 350° C. in contact with the precursors and oxygen for a time sufficient to form a film of TiO₂—Ta₂O₅ on the surface of the device of at least 50 nm. Preferably the thickness is 50-75 nm. Optionally, the device is post-annealed in air at 600° C. for at least 10 minutes. The resulting film has an average composition of 2 at % TiO₂-98 at % Ta₂O₅+/−1 at % TiO₂.

Example 8: An invasive surgical device is placed in a vacuum deposition chamber at a reduced pressure of 1 Torr and exposed to a mixture of Ti(NMe₂)₄ and Ta(NMe₂)₅ in a ratio of 10:1 in an ALD mode. The device is held at a temperature of 250° C. and exposed to alternating pulses of Ti(NMe₂)₄ mixed with Ta(NMe₂)₅ in the specified ratio and water vapor, each separated by an inert gas purge for 1100 cycles sufficient to form a film of TiO₂—Ta₂O₅ on the surface of the device of 54 nm+/−5 nm with an overall composition of 94 at % TiO₂-6 at % Ta₂O₅+/−2 at % TiO₂. Optionally, the device is post-annealed in air at 600° C. for at least 10 minutes.

Example 9: An invasive surgical device is placed in a vacuum deposition chamber at a reduced pressure of 1 Torr and exposed to a mixture of Ti(NMe₂)₄ and Ta(NMe₂)₅ in a ratio of 5:1 in an ALD mode. The device is held at a temperature of 250° C. and exposed to alternating pulses of Ti(NMe₂)₄ mixed with Ta(NMe₂)₅ in the specified ratio and water vapor, each separated by an inert gas purge for 950 cycles sufficient to form a film of TiO₂—Ta₂O₅ on the surface of the device of 52 nm+/−5 nm with an overall composition of 89 at % TiO₂-11 at % Ta₂O₅+/−2 at % TiO₂. Optionally, the device is post-annealed in air at 600° C. for at least 10 minutes.

Example 10: An invasive surgical device is placed in a vacuum deposition chamber at a reduced pressure of 1 Torr and exposed to a mixture of Ti(NMe₂)₄ and Ta(NMe₂)₅ in a ratio of 1:1 in an ALD mode. The device is held at a temperature of 250° C. and exposed to alternating pulses of Ti(NMe₂)₄ mixed with Ta(NMe₂)₅ in the specified ratio and water vapor, each separated by an inert gas purge for 800 cycles sufficient to form a film of TiO₂—Ta₂O₅ on the surface of the device of 52 nm+/−5 nm with an overall composition of 59 at % TiO₂-41 at % Ta₂O₅+/−2 at % TiO₂. Optionally, the device is post-annealed in air at 600° C. for at least 10 minutes.

Example 11: An invasive surgical device is placed in a vacuum deposition chamber at a reduced pressure of 1 Torr and exposed to a mixture of Ti(NMe₂)₄ and Ta(NMe₂)₅ in a ratio of 1:10 in an ALD mode. The device is held at a temperature of 250° C. and exposed to alternating pulses of Ti(NMe₂)₄ mixed with Ta(NMe₂)₅ in the specified ratio and water vapor, each separated by an inert gas purge for 775 cycles to form a film of TiO₂—Ta₂O₅ on the surface of the device of 58 nm+/−5 nm with an overall composition of 14 at % TiO₂-86 at % Ta₂O₅+/−2 at % TiO₂. Optionally, the device is post-annealed in air at 600° C. for at least 10 minutes.

Example 12: An invasive surgical device is placed in a vacuum deposition chamber at a reduced pressure of 1 Torr and exposed to Ta(NMe₂)₅ in an ALD mode. The device is held at a temperature of 250° C. and exposed to alternating pulses of Ta(NMe₂)₅ and water vapor, each separated by an inert gas purge for 650 cycles to form a film of Ta₂O₅ on the surface of the device of 52 nm+/−5 nm. Optionally, the device is post-annealed in air at 600° C. for at least 10 minutes.

Example 13: An invasive surgical device is placed in a vacuum deposition chamber at a reduced pressure of 1 Torr and exposed to Ti(NMe₂)₄ and Ta(NMe₂)₅ in alternating doses in a ratio of 50:1 in an ALD mode. The device is held at a temperature of 250° C. and exposed to alternating pulses of Ti(NMe₂)₄ and Ta(NMe₂)₅ in the specified ratio and water vapor, each separated by an inert gas purge for 1020 cycles to form a film of TiO₂—Ta₂O₅ on the surface of the device of 52 nm+/−5 nm with an overall composition of 99% TiO₂-1 at % Ta₂O₅+/−0.5 at % TiO₂. Optionally, the device is post-annealed in air at 600° C. for at least 10 minutes.

Example 14: An invasive surgical device is placed in a vacuum deposition chamber at a reduced pressure of 1 Torr and exposed to a mixture of Ti(NMe₂)₄ and Ta(NMe₂)₅ in a ratio of 10:1 in an ALD mode. The device is held at a temperature of 250° C. and exposed to alternating pulses of Ti(NMe₂)₄ mixed with Ta(NMe₂)₅ in the specified ratio and water vapor, each separated by an inert gas purge for 1100 cycles. During the process, an ALD cycle of Ce using Ce(iPrCp)₂-isopropylamindinate and water is introduced at an interval of once every 100 Ta/Ti cycles to form a film of TiO₂—Ta₂O₅ on the surface of the device of 54 nm+/−5 nm with an overall composition of 93 at % TiO₂-6 at % Ta₂O₅-0.5 at % CeO₂+/−2 at % TiO₂, +/−0.1 at % CeO₂. Optionally, the device is post-annealed in air at 600° C. for at least 10 minutes.

Example 15: A TiO₂ layer is formed on an invasive medical device by ALD and subsequently a patterned Ta₂O₅ layer is deposited onto it by ALD using the following process. The substrate may be a metal, a ceramic, or a polymer. The invasive surgical device is placed in a vacuum deposition chamber at a reduced pressure of 1 Torr and exposed to Ti(NMe₂)₄ at a temperature of 250° C. and exposed to alternating pulses of Ti(NMe₂)₄ and water vapor, each separated by an inert gas purge for 1000 cycles. A TiO₂ layer of 50 nm+/−2 nm is formed. The TiO₂ phase mixture is rutile and anatase with >60% anatase by volume. A PS-b-PMMA BCP is applied to the TiO₂ layer and cured. The PMMA portion of the BCP is removed using a solvent containing a portion of acetic acid. Ta₂O₅ is then selectively deposited on the exposed TiO₂ layer by ALD using Ta(NMe₂)₅ at 1 Torr and 250° C. Alternating cycles of Ta(NMe₂)₅ and water are used separated by inert gas purges of nitrogen for a total of 12 cycles to deposit a Ta₂O₅ film of 1 nm+/−0.3 nm. After deposition, the remaining BCP (i.e., PS) is removed by oxygen ashing. The resulting structure has lamellar regions of Ta₂O₅ of 40 nm+/−10 nm width separated by 40 nm+/10 nm spaces disposed on the TiO₂ layer giving a height to width aspect ratio of 0.025:1.

Example 16: A TaO₅ layer is formed on an invasive medical device by ALD and subsequently a patterned TiO₂ layer is deposited onto it by ALD using the following process. The substrate may be a metal, a ceramic, or a polymer. The invasive surgical device is placed in a vacuum deposition chamber at a reduced pressure of 1 Torr and exposed to Ta(NMe₂)₅ at a temperature of 250° C. and exposed to alternating pulses of Ta(NMe₂)₅ and water vapor, each separated by an inert gas purge for 250 cycles. A Ta₂O₅ layer of 20 nm+/−2 nm is formed. The Ta₂O₅ is amorphous. A PS-b-PMMA BCP is applied to the Ta₂O₅ layer and cured. The PMMA portion of the BCP is removed using a solvent containing a portion of acetic acid. TiO₂ is then selectively deposited on the exposed Ta₂O₅ layer by ALD using TiCl₄ at 1 Torr and 250° C. Alternating cycles of TiCl₄ and water are used separated by inert gas purges of nitrogen for a total of 500 cycles to deposit a TiO₂ film of 20 nm+/−5 nm. After deposition, the remaining BCP (i.e., PS) is removed by oxygen ashing. The resulting structure has lamellar regions of TiO₂ of 40 nm+/−10 nm width separated by 40 nm+/10 nm spaces disposed on the Ta₂O₅ layer giving a height to width aspect ratio of 0.5:1. The TiO₂ regions are >50% anatase by volume.

Example 17: A TiO₂ layer doped with CeO₂ is formed on an invasive medical device by ALD and subsequently a patterned Ta₂O₅ layer is deposited onto it by ALD using the following process. The substrate may be a metal, a ceramic, or a polymer. The invasive surgical device is placed in a vacuum deposition chamber at a reduced pressure of 1 Torr and exposed to Ti(NMe₂)₄ at a temperature of 250° C. and exposed to alternating pulses of Ti(NMe₂)₄ and water vapor, each separated by an inert gas purge for 1000 cycles. A Ce cycle using Ce(iPrCp)₂-isopropylamindinate and water is introduced at an interval of once every 200 TiO₂ cycles. A TiO₂ layer of 50 nm+/−2 nm containing an average of 0.1-0.5 at % CeO₂ is formed. The deposited layer and device is annealed at 450° C. for 2 hr. The TiO₂ phase mixture is rutile and anatase with >50% anatase by volume. A PS-PMMA BCP is applied to the TiO₂ layer and cured. The PMMA portion of the BCP is removed using a solvent containing a portion of acetic acid. Ta₂O₅ is then selectively deposited on the exposed TiO₂ layer by ALD using Ta(NMe₂)₅ at 1 Torr and 250° C. Alternating cycles of Ta(NMe₂)₅ and water are used separated by inert gas purges of nitrogen for a total of 12 cycles to deposit a Ta₂O₅ film of 1 nm+/−0.3 nm. After deposition, the remaining BCP (i.e., PS) is removed by oxygen ashing. The resulting structure has lamellar regions of Ta₂O₅ of 40 nm+/−10 nm width separated by 40 nm+/10 nm spaces disposed on the TiO₂ layer giving a height to width aspect ratio of 0.025:1.

Thin film coatings thus formed have combined antimicrobial and improved osseointegrative properties that they impart to the medical device. Prior to placement, the medical device may be irradiated with light to produce the desired antimicrobial effect. The illumination may be UV or visible light. The illumination may be provided by the ambient light in the operating room or by an ancillary light. The light may comprise light emitting diodes (LEDs) with wavelengths from 360-410 nm.

The subject invention may be embodied in the forgoing examples that are by no means restrictive, but intended to illustrate the invention. Any embodiment herein described may be combined with any other embodiment described, in particular methods of patterning the films with film deposition examples and with different substrates.

What is claimed is:

1. An invasive surgical device, comprising a thin film coating on at least a portion of the exterior surface of the device, the thin film coating comprising Ta₂O₅ with 0-99.9 at % TiO₂ and a conformality greater than 50%.

2. The device of claim 1, where the thin film coating is between 10 and 1000 nm thick.

3. The device of claim 1, where the thin film coating is less than 150 nm thick.

4. The device of claim 1, where the thin film coating has an average composition of greater than 1 at % Ta₂O₅.

5. The device of claim 1, where the device is comprised of polyetheretherketone.

6. The device of claim 1, where the average cerium content of the thin film coating is greater than 0.1 at %.

7. The device of claim 1, where the thin film coating comprises phase regions of at least two of TiO₂, Ta₂O₅, and TiTa₂O₇.

8. The device of claim 1, where the conformality of the thin film coating is greater than 90%.

9. The device of claim 8 where the thin film coating is between 10 and 1000 nm thick.

10. The device of claim 8 where the thin film coating is less than 150 nm thick.

11. The device of claim 8 where the thin film coating has an average composition of greater than 1 at % Ta₂O₅.

12. The device of claim 8 where the device is comprised of polyetheretherketone.

13. The device of claim 8 where the average cerium content of the thin film coating is greater than 0.1 at %.

14. The device of claim 8 where the thin film coating comprises phase regions of at least two of TiO₂, Ta₂O₅, and TiTa₂O₇.

15. An invasive surgical device, comprising a thin film coating on at least a portion of the exterior surface of the device, the thin film coating having a layer comprising TiO₂ and a layer comprising Ta₂O₅ where a first of the two layers is a continuous layer, a second of the two layers is a discontinuous layer having regions separated by spaces, and the second layer is deposited on the first layer.

16. The invasive surgical device of claim 15 where the thickness of the continuous layer is less than 100nm and the thickness of the discontinuous layer is less than 100 nm.

17. The invasive surgical device of claim 15 where the average spacing between the regions of the discontinuous layer is less than 100 nm.

18. The invasive surgical device of claim 15 where the continuous layer comprises TiO₂ and the discontinuous layer comprises Ta₂O₅.

19. The invasive surgical device of claim 18 where the thickness of the continuous layer is between 20-60 nm, the spacing of the regions in the discontinuous layer is greater than 25 nm and the height of the regions in the discontinuous layer is less than 5 nm.

20. The invasive surgical device of claim 15 where the continuous layer comprises Ta₂O₅ and the discontinuous layer comprises TiO₂.
